(19) United States
(12) Patent Application Publication
(10) Pub. No.: US 2013/0183746 A1
(43) Pub. Date: Jul. 18, 2013

(54) BIO-CHIP MODULE AND DEVICE FOR MEASURING BIO-CHIP

(71) Applicants: Dong Woo Lee, Suwon (KR); Bo Sung Ku, Suwon (KR)

(72) Inventors: Dong Woo Lee, Suwon (KR); Bo Sung Ku, Suwon (KR)

(73) Assignee: SAMSUNG ELECTRO-MECHANICS CO., LTD.

(21) Appl. No.: 13/409,939

(22) Filed: Mar. 1, 2012

(30) Foreign Application Priority Data
Jan. 17, 2012 (KR) 10-2012-0005141

(51) Int. Cl. C12M 1/34; C12M 1/00

(52) U.S. Cl. 435/287.1; 435/283.1

(57) ABSTRACT

There is provided a bio-chip module including: a first substrate including a plurality of projections formed on a first face thereof and made of a plastic material, the plurality of projections having a bio-material attached thereto; and a second substrate including a plurality of through holes into which the projections are inserted, the second substrate being combined with the first face of the first substrate.

### CROSS-REFERENCE TO RELATED APPLICATIONS

This application claims the priority of Korean Patent Application No. 10-2012-0005141 filed on Jan. 17, 2012, in the Korean Intellectual Property Office, the disclosure of which is incorporated herein by reference.

### BACKGROUND OF THE INVENTION

1. Field of the Invention

The present invention relates to a bio-chip module and a device for measuring a bio-chip and, more particularly, to a bio-chip module and a device for measuring a bio-chip capable of reducing measurement errors in measuring a bio-material attached to a bio-chip.

2. Description of the Related Art

Recently, demand for a bio-technology apparatus such as a bio-medical device able to quickly diagnose various human diseases has increased. Thus, the development of a bio-sensor or a bio-chip capable of rapidly running a diagnostic test for a particular disease, which has conventionally taken a long period of time in a hospital or laboratory, has been undertaken.

In general, a bio-chip is made of a glass material. However, it is very difficult to process and mold a bio-chip made of a glass material, and unit manufacturing costs are high. Also, since a bio-chip made of a glass material may be easily damaged, it is inconvenient and relatively difficult to handle.

Thus, the development of a bio-chip whose unit manufacturing costs are low and which can be easily handled has been required.

Meanwhile, in order to resolve such shortcomings, attempts have been made to manufacture a bio-chip by using a different material. However, a bio-chip made of any material other than a glass material can be easily deformed when used or kept in storage, causing a problem in which precise measurement results of a bio-material may not be anticipated.

### SUMMARY OF THE INVENTION

An aspect of the present invention provides a bio-chip module which incurs low unit manufacturing costs and can be easily handled.

Another aspect of the present invention provides a bio-chip module and a device for measuring a bio-chip capable of minimizing a measurement error.

According to an aspect of the present invention, there is provided a bio-chip module including: a first substrate including a plurality of projections formed on a first face thereof and made of a plastic material, the plurality of projections having a bio-material attached thereto; and a second substrate including a plurality of through holes into which the projections are inserted, the second substrate being combined with the first face of the first substrate.

The second substrate may be made of a metallic material or a ceramic material.

The bio-chip module may further include a third substrate installed on a second face of the first substrate such that the first face of the first substrate is tightly attached to one face of the second substrate.

The second substrate may be made of a metallic material or a magnetic material, and the third substrate may be made of a material different from that of the second substrate, among the metallic material and the magnetic material.

The third substrate may include a recess formed on one face thereof and allowing the first substrate to be inserted thereinto.

Each of the through holes has a sloped portion on an edge thereof.

According to another aspect of the present invention, there is provided a device for measuring a bio-chip including: a bio-chip module including a first substrate including a plurality of projections formed on a first face thereof, the plurality of projections having a bio-material attached thereto, and a second substrate installed on the first face of the first substrate and including a plurality of through holes into which the projections of the first substrate are inserted; and a measurement unit measuring the bio-material attached to the projections.

The bio-chip module may further include a third substrate installed on a second face of the first substrate such that the first face of the first substrate is tightly attached to one face of the second substrate.

The second substrate may be made of a metallic material or a magnetic material, and the third substrate may be made of a material different from that of the second substrate, among the metallic material and the magnetic material.

The device may further include a flattening member evenly distributing pressure of the third substrate on the first substrate.

The device may further include a pressing unit pressing the second face of the first substrate such that the first substrate is flattened in a state of being combined with the second substrate.

The device may further include a prop member supporting the first substrate such that a predetermined distance is maintained between the bio-material attached to the projections and the measurement unit.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is an exploded perspective view of a bio-chip module according to an embodiment of the present invention;

FIG. 2 is a perspective view of the bio-chip module illustrated in FIG. 1;

FIG. 3 is a cross-sectional view taken along line A-A in the bio-chip module illustrated in FIG. 2;

FIG. 4 is an enlarged view of a portion B illustrated in FIG. 3;

FIG. 5 is an enlarged view of a portion B for explaining a bio-chip module according to another embodiment of the present invention;

FIG. 6 is an exploded perspective view of a bio-chip module according to another embodiment of the present invention;

FIG. 7 is a perspective view of the bio-chip module illustrated in FIG. 6;

FIG. 8 is a cross-sectional view taken along line C-C of bio-chip module illustrated in FIG. 7;

FIG. 9 is a view showing a device for measuring a bio-chip and a bi-chip module mounted in the device for measuring a bio-chip according to an embodiment of the present invention;

FIG. 10 is a view showing a device for measuring a bio-chip and a bi-chip module mounted in the device for measuring a bio-chip according to another embodiment of the present invention;

FIG. 11 is a graph showing scan values using the related art bio-chip module; and

FIG. 12 is a graph showing scan values using the bio-chip module according to an embodiment of the present invention.

### DETAILED DESCRIPTION OF THE EMBODIMENTS

Exemplary embodiments of the present invention will now be described in detail with reference to the accompanying drawings.

The invention may, however, be embodied in many different forms and should not be construed as being limited to the embodiments set forth herein. Rather, these embodiments are provided so that this disclosure will be thorough and complete, and will fully convey the scope of the invention to those skilled in the art.

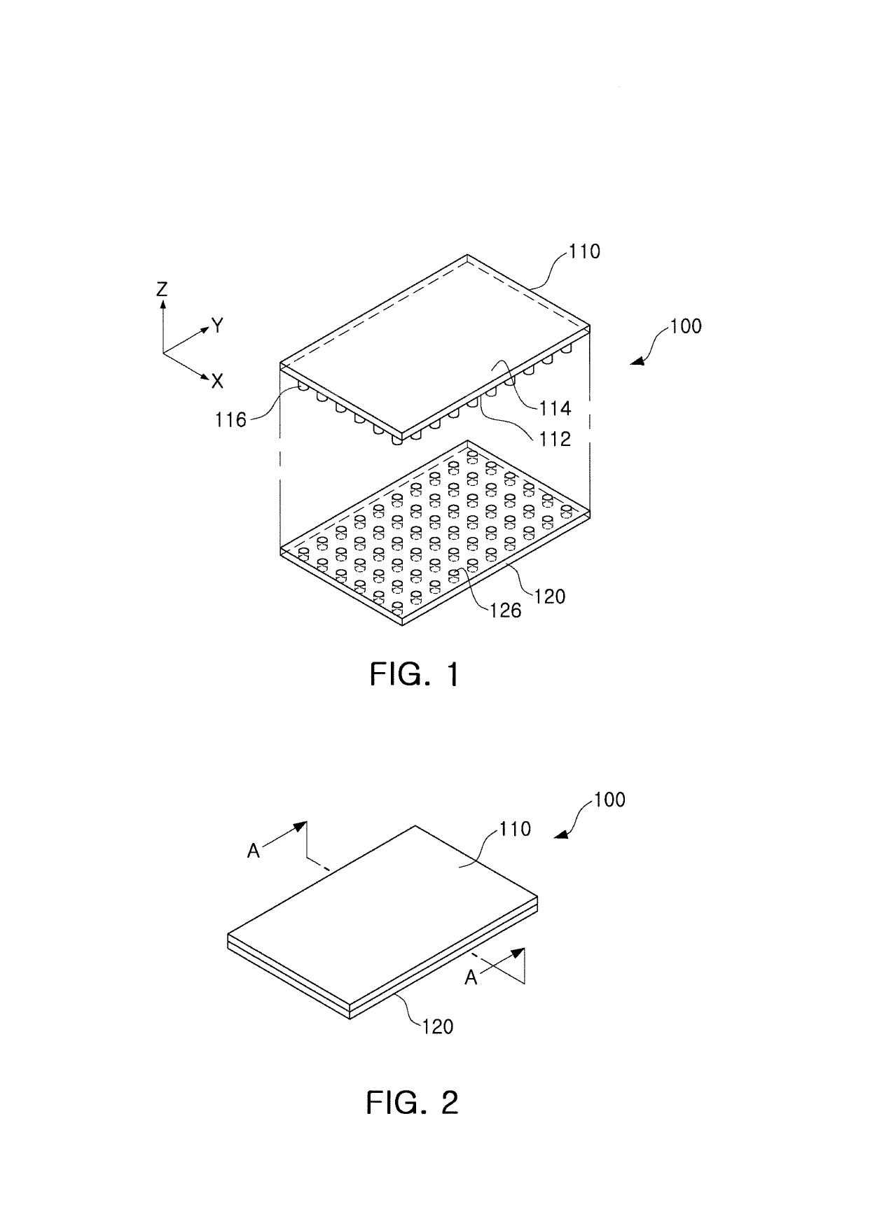

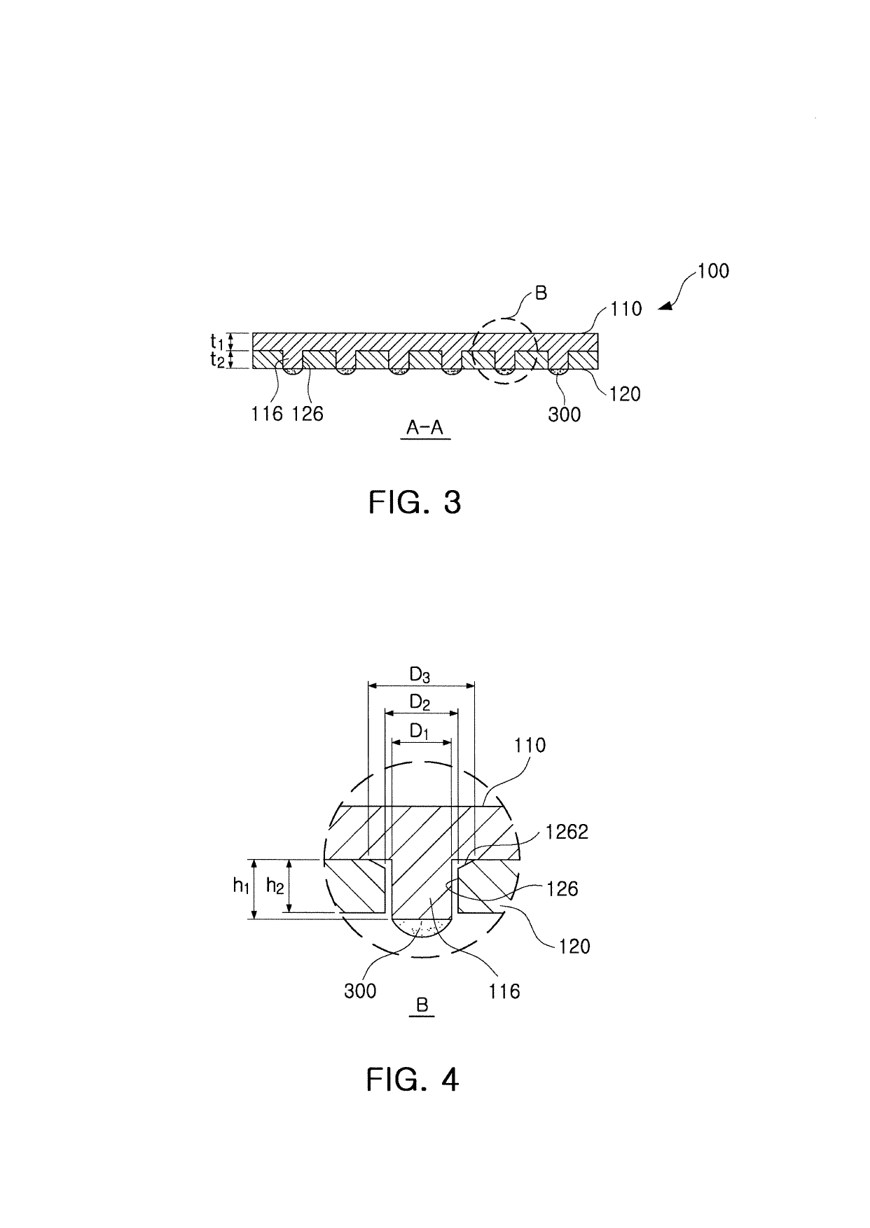

FIG. 1 is an exploded perspective view of a bio-chip module according to an embodiment of the present invention. FIG. 2 is a perspective view of the bio-chip module illustrated in FIG. 1. FIG. 3 is a cross-sectional view taken along line A-A in the bio-chip module illustrated in FIG. 2. FIG. 4 is an enlarged view of a portion B illustrated in FIG. 3.

A bio-chip module 100 according to an embodiment of the present invention includes a first substrate 110 and a second substrate 120.

The first substrate 110 may have a shape of a substantially thin plate, and may be used as a bio-chip to which a bio-material is attached. To this end, the first substrate 110 may include a plurality of projections 116 formed on a first face 112. In detail, the plurality of projections 116 may be formed to extend in a downward direction (i.e., −Z axis direction) from the first face 112 of the first substrate 110. All of the plurality of projections 116 may have the same length and may be arranged at regular intervals along an X axis and a Y axis. Each of the projections 116 may have a section having a circular, quadrangular, or polygonal shape. End portions of the projections 116 may be processed to be rough to facilitate an attachment of a bio-material thereto or an auxiliary material assisting in an attachment of a bio-material may be coated on the end portions of the projections 116. A second face 114 of the first substrate 110 may be a plane provided in X-Y directions as depicted in FIG. 1.

Meanwhile, the first substrate 110 may be made of a plastic material. Here, since the first substrate 110 made of a plastic material can be mass-produced using a mold, unit manufacturing costs can be reduced as compared with a bio-chip made of a glass material. In addition, the first substrate 110 made of a plastic material is relatively light-weight and has relatively low brittleness as compared with the bio-chip made of a glass material, so it may be easily handled and may suffer a low incidence of damage due to careless handling.

The second substrate 120 may substantially have a shape of a plate, and have a plurality of through holes 126. The through holes 126 are formed to penetrate vertically through the second substrate 120 in the Z axis direction as depicted in FIG. 1, and may be formed in the second substrate 120 at the same intervals as those of the projections 116. The second substrate 120 may be thicker than the first substrate 110. Namely, the thickness t2 of the second substrate 120 may be larger than the thickness t1 of the first substrate 110 (See FIG. 3). This structure can prevent the first substrate 110 from being deformed, namely, from being bent by virtue of the second substrate 120. The second substrate 120 may be made of a metallic material or a magnetic material. Alternatively, the second substrate 120 may be made of a material (e.g., ceramic) other than a metallic material having low deformation rate as compared with the material of the first substrate 110.

Meanwhile, FIG. 1 illustrates that the first substrate 110 and the second substrate 120 have the same size, but the sizes of the first substrate 110 and the second substrate 120 may be different as necessary. For example, the first substrate 110 may be larger than the second substrate 120, or the second substrate 120 may be larger than the first substrate 110.

As shown in FIG. 2, the first substrate 110 and the second substrate 120 are vertically combined to form a bio-chip module 100. Specifically, after a bio-material is attached to the plurality of projections 116, the first substrate 110 may be combined with the second substrate 120. However, in a state in which the first and second substrates 110 and 120 are combined, a bio-material may be attached to the projections 116 of the first substrate 110.

A sectional structure of the bio-chip module 100 will now be described with reference to FIGS. 3 and 4.

The projections 116 may be inserted into the corresponding through holes 126 when the first and second substrates 110 and 120 are combined. Here, the end portions of the projections 116 or a bio-material 300 attached to the projections 116 may be exposed to the outside (i.e., downwardly) of the through holes 126. Such a structure may be advantageous in inspecting the bio-material 300 by using a measurement unit.

The diameter D2 of the through hole 126 may be equal to the diameter D1 of the projection 116 or may be larger than the diameter D1 of the projection 116. In the former case, a binding strength between the projection 116 and the through hole 126 is high, so cohesion between the first and second substrates 110 and 120 is excellent. Meanwhile, in the latter case, since the projections 116 can be easily inserted into the through holes 126, the first and second substrates 110 and 120 can be easily combined.

In addition, as shown in FIG. 4, a sloped portion 1262 may be formed on an edge of the through hole 126. The sloped portion 1262 may be formed on one face, of the second substrate 120, facing the first substrate 110. The sloped portion 1262 may be formed by filleting or chamfering the edge of the through hole 126. The sloped portion 1262 allows the projection 116 to be easily inserted into the through hole 126.

In the bio-chip module 100 having the structure as described above, since the second substrate 120 which is relatively solid and has low strain is combined with the first substrate 110, deformation of the first substrate 110, when used, may be minimized. According to the present embodiment, since the first substrate 110 is restrained from being deformed by the second substrate 120, the first substrate 110 may be made of a plastic material which is lightweight and easily handled.

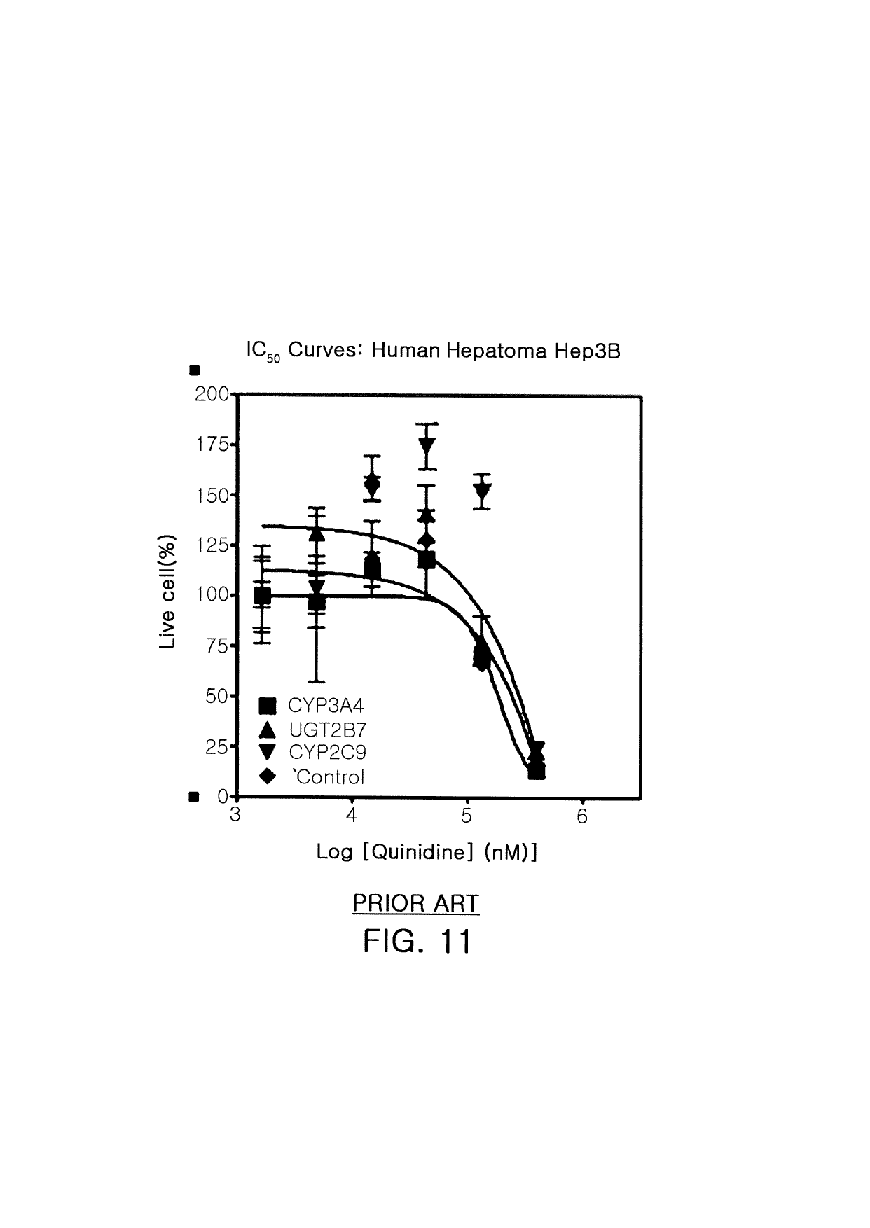

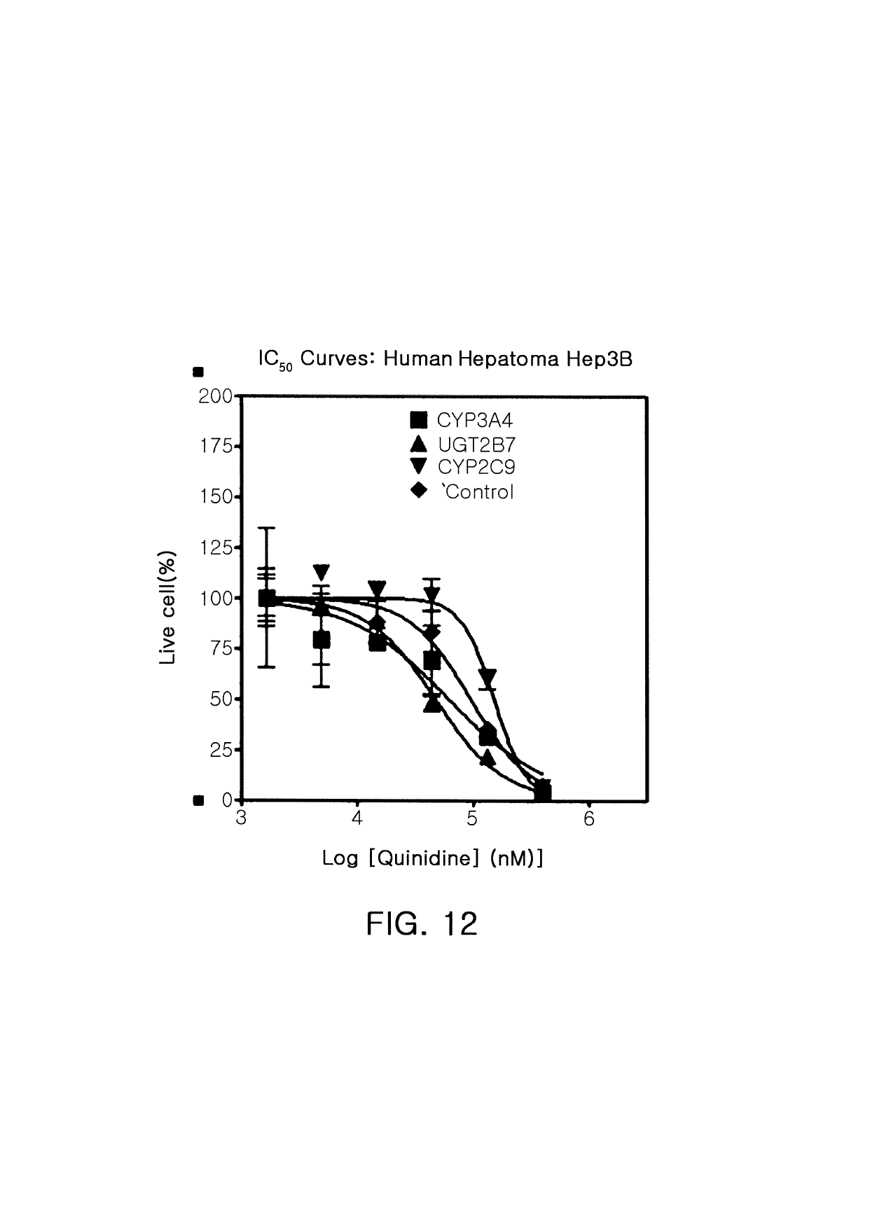

Meanwhile, in order to confirm the effect of the bio-chip module 100 according to an embodiment of the present invention, measurement was made by attaching the same liver cancer cells to the related art bio-chip module and the bio-chip module 100 according to the embodiment of the present invention, respectively. FIG. 11 is a graph showing scan values using the related art bio-chip module, and FIG. 12 is a graph showing scan values using the bio-chip module according to an embodiment of the present invention.

As shown in FIG. 11, the results showed that measurement values using the related art bio-chip module are significantly outside of ideal dose response curves, while the measurement values using the bio-chip module 100 according to the embodiment of the present invention are within the ideal dose response curves.

This means that, in the bio-chip module 100 according to the present embodiment, similar results were derived over the entirety of the identical liver cancer cells regardless of their positions on the bio-chip, but in the related art bio-chip module, different results were derived according to the positions of the liver cancer cells on the bio-chip in spite of their being identical liver cancer cells.

Namely, it was ascertained that the use of the bio-chip module 100 according to the embodiment of the present invention can obtain ideal scan values with respect to a measurement target material.

Other embodiments of the present invention will now be described. For reference, the same reference numerals will be used for the same elements as those of the former embodiment, and a detailed description thereof will be omitted.

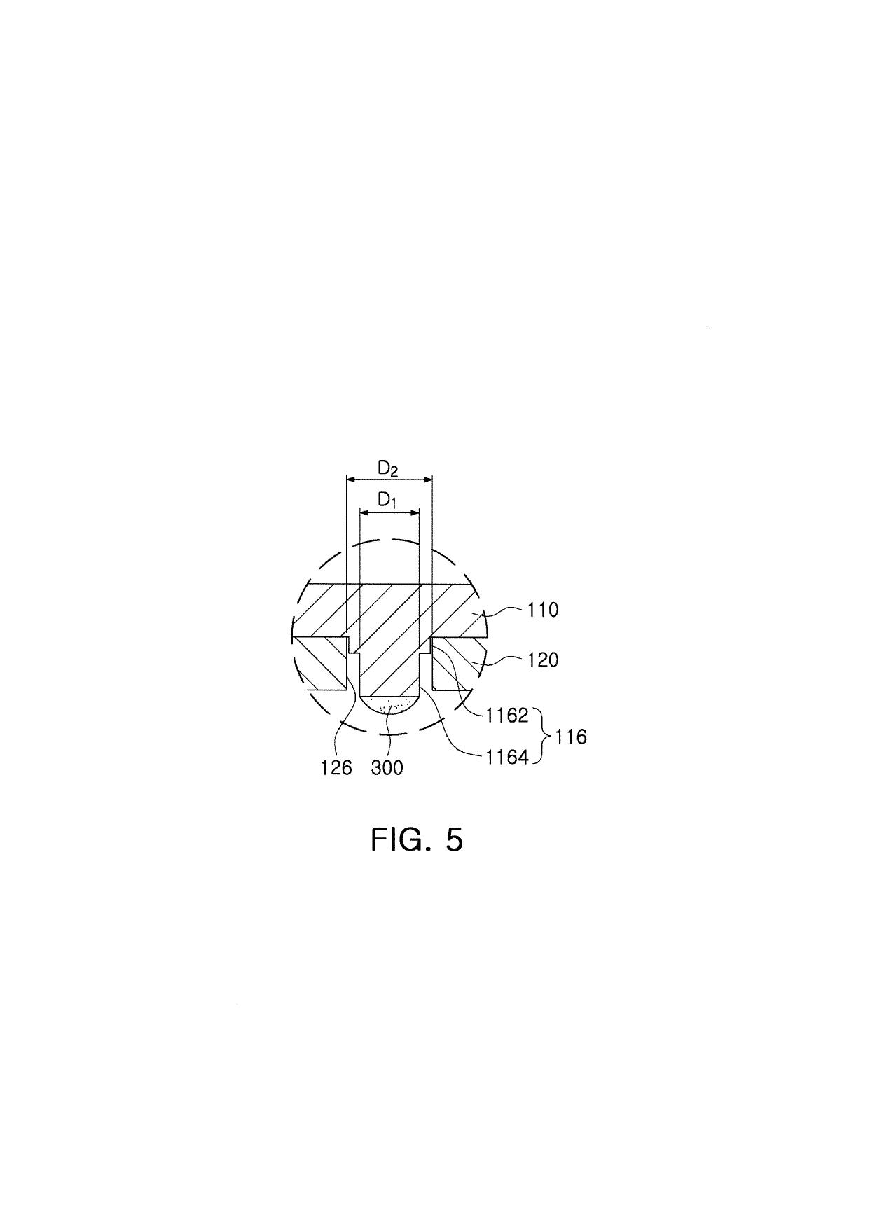

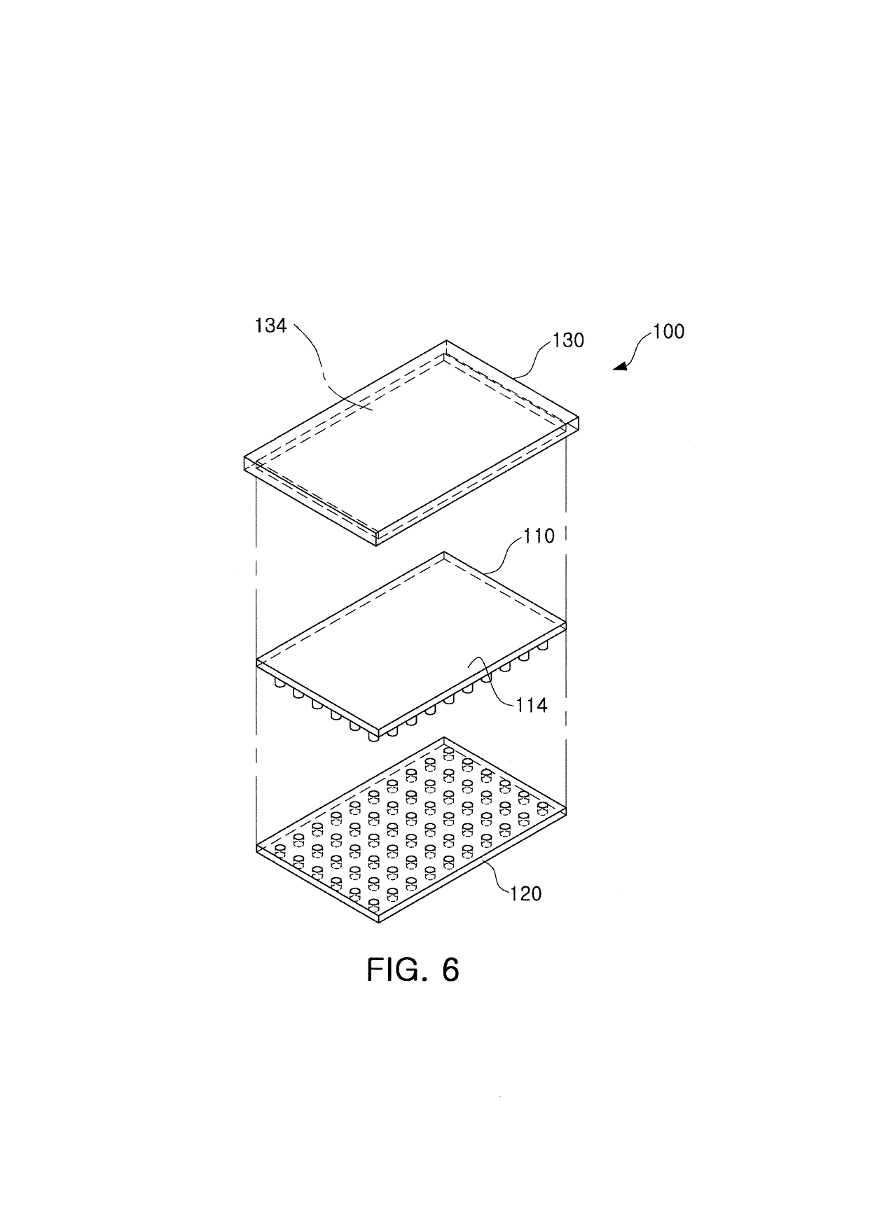

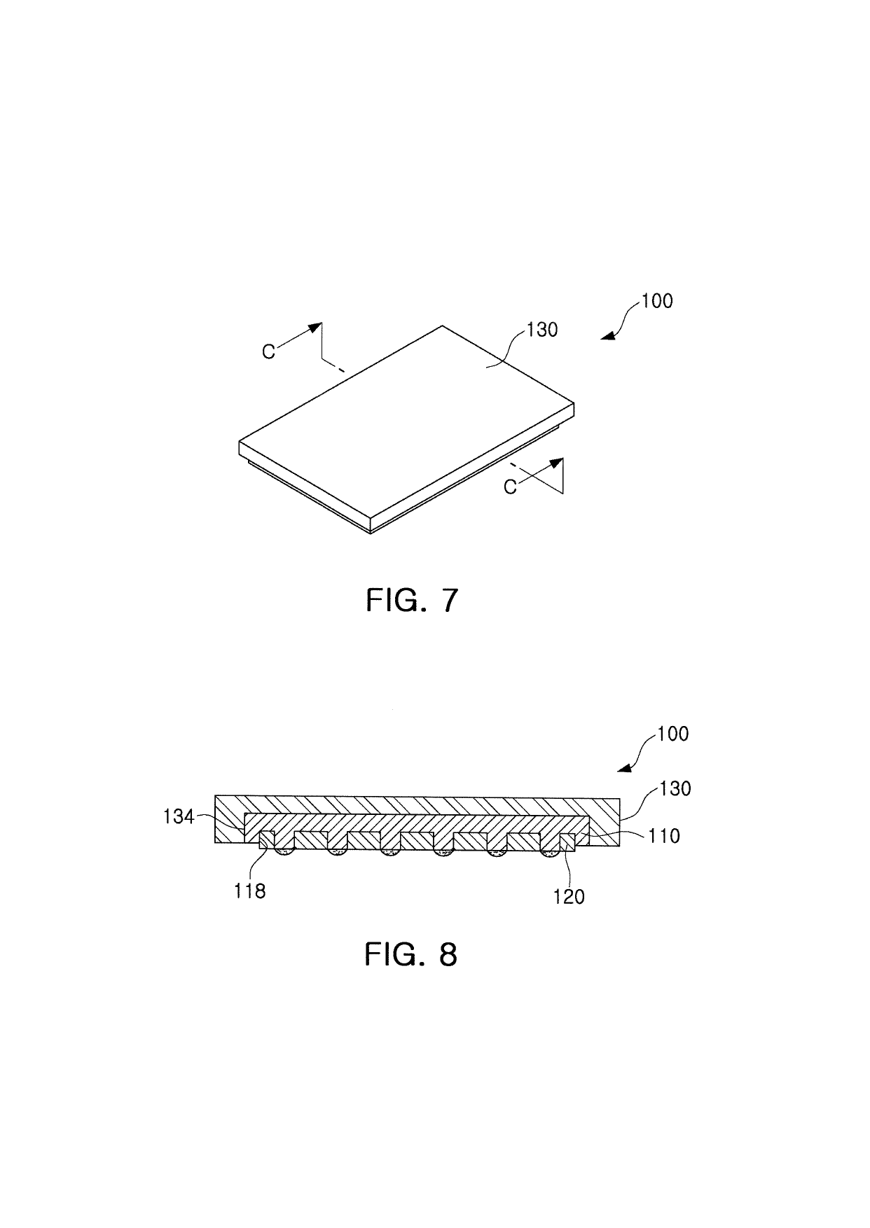

FIG. 5 is an enlarged view of a portion B for explaining a bio-chip module according to another embodiment of the present invention. FIG. 6 is an exploded perspective view of a bio-chip module according to another embodiment of the present invention. FIG. 7 is a perspective view of the bio-chip module illustrated in FIG. 6. FIG. 8 is a cross-sectional view taken along line C-C of the bio-chip module illustrated in FIG. 7.

The bio-chip module 100 according to another embodiment of the present invention will now be described with reference to FIG. 5. The bio-chip module 100 according to another embodiment of the present invention is different from the bio-chip module 100 of the former embodiment, in the shape of the projection 116.

As shown in FIG. 5, the projection 116 may be divided into a first projection portion 1162 and a second projection portion 1164 having different diameters. Namely, the first projection portion 1162 may have the substantially same diameter as the diameter D2 of the through hole 126, and the second projection portion 1164 may have the diameter D1 smaller than the diameter D2 of the through hole 126. Here, the first projection portion 1162 may increase a binding strength between the first substrate 110 and the second substrate 120, and the second projection portion 1164 may provide a place to which the bio-material 300 is to be attached.

Such a structure can prevent the bio-material 300 from being undesirably brought into contact with the through hole 126, without degrading the binding force between the first substrate 110 and the second substrate 120. Namely, in the present embodiment, the bio-material 300 attached to the projection 116 can be prevented from contaminating an inner wall of the through hole 126 or from coming into contact with the inner wall of the through hole 126 so as to thus be deformed or released, when the first substrate 110 and the second substrate 120 are combined.

The bio-chip module 100 according to another embodiment of the present invention will be described with reference to FIGS. 6, 7, and 8. The bio-chip module 100 according to this embodiment is different from the bio-chip module 100 according to the former embodiments, in that it further includes a third substrate 130.

The third substrate 130 may substantially have a shape of a plate and may be installed on the second face 114 of the first substrate 110. The third substrate 130 may have a recess 134 formed on one face thereof. The recess 134 may have a size allowing the first substrate 110 to be inserted therein, and may have a depth allowing the first substrate 110 to be completely accommodated therein (See FIG. 8).

In addition, the third substrate 130 may be made of a material (e.g., a metallic material) having a certain mass in order to evenly apply uniform pressure to the second face 114 of the first substrate 110.

In particular, in the bio-chip module 100 according to the present embodiment, the second substrate 120 may be made of a metallic material and the third substrate 130 may be made of a magnetic material. On the contrary, the second substrate 120 may be made of a magnetic material and the third substrate 130 may be made of a metallic material.

In such a structure, pressure according to a self-load of the third substrate 130 and magnetic attraction acting between the second substrate 120 and the third substrate 130 can evenly act on both faces of the first substrate 110. Thus, in the bio-chip module 100 according to the present embodiment, the first substrate 110 can be effectively restrained from being deformed, and deformation of the first substrate 110 can be corrected. Thus, the bio-chip module 100 having such an effect can be advantageously used for a bio-chip having a relatively large area.

Meanwhile, a flattening member (e.g., a member made of a glass or ceramic material) may be further provided between the third substrate 130 and the first substrate 110. The addition of the flattening member can maximize a correction effect of the first substrate 110 through the flattening member and the attraction between the second substrate 120 and the third substrate 130.

For reference, reference numeral 118 denotes a recess formed in the first substrate 110 and allowing the second substrate 120 to be inserted therein.

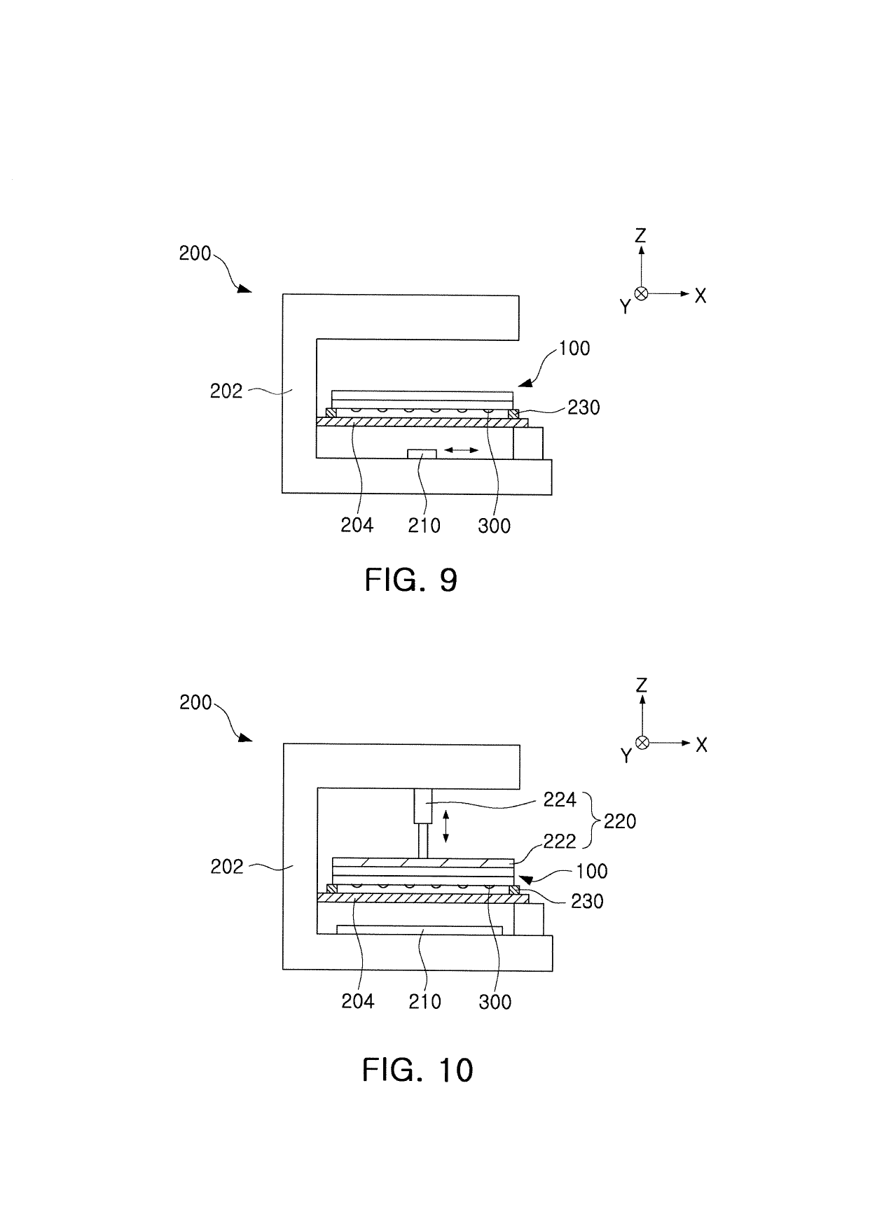

A device for measuring a bio-chip will be described with reference to FIGS. 9 and 10. FIG. 9 is a view showing a device for measuring a bio-chip and a bio-chip module mounted in the device for measuring a bio-chip according to an embodiment of the present invention, and FIG. 10 is a view showing a device for measuring a bio-chip and a bi-chip module mounted in the device for measuring a bio-chip according to another embodiment of the present invention.

As shown in FIG. 9, a device 200 for measuring a bio-chip according to an embodiment of the present invention may include a measurement device body 202, a support plate 204, a measurement unit 210, and a prop member 230.

The measurement device body 202 may dictate an external appearance of the measurement device, and may further include a control unit (not shown) controlling units other than the measurement unit 210.

The support plate 204 may be installed above the measurement unit 210, and provide a place in which the bio-chip module 100 can be mounted. The support plate 204 may be made of a transparent material allowing light irradiated from the measurement unit 210 and light reflected from the bio-material 300 to pass therethrough.

The measurement unit 210 may be installed in the measurement device body 202, and may measure constituents, or the like, of the bio-material 300 attached to the bio-chip module 100. The measurement unit 210 may move in X axis and Y axis directions as shown in FIG. 9 in the measurement device body 202 in order to measure the bio-materials 300 attached to the projections 116. To this end, the measurement device body 202 may further include a transfer (or moving) unit.

The prop member 230 may be installed on the support plate 204. The prop member 230 may support the bio-chip module 100. In detail, the prop member 230 may uniformly maintain a distance between the bio-material 300 attached to the projections 116 and the measurement unit 210. In addition, the prop member 230 may maintain the distance between the bio-material 300 and the support plate 204 such that the bio-material 300 is prevented from coming into contact with the support plate 204.

As illustrated in FIG. 10, the device 200 for measuring a bio-chip according to another embodiment of the present invention may further include a pressing unit 220.

The pressing unit 220 according to the present embodiment may be installed in the measurement device body 202 and may move up and down in a Z axis direction as shown in FIG. 10. The pressing unit 220 may include a flattening member 222 and a cylinder 224. The flattening member 222 may have one face (i.e., a lower face in FIG. 10) provided in X-Y directions, and may be made of alloy steel, or the like, having very low strain. The cylinder 224 is installed in the measurement device body 202 and may move the flattening member 222 up and down in the Z axis direction.

In a state in which the bio-chip module 100 is mounted in the measurement device body 202, the pressing unit 220 having the above-described configuration may press one face (an upper face in FIG. 10) of the bio-chip module 100 with a certain amount of pressure.

Thus, the device 200 for measuring a bio-chip according to the present embodiment may further tightly attach the first substrate 110 to the second substrate 120, and correct the deformation of the first substrate 110 such that the first substrate 110 may be flattened based on the second substrate 120.

For reference, the pressing unit 220 according to the present embodiment may be made of a magnetic material or a metallic material enabling attractive force with the second substrate 120. In this case, the third substrate 130 of the bio-chip module 100 may be omitted.

As set forth above, in a bio-chip module and a device for measuring a bio-chip according to embodiments of the invention, since a distance between a bio-material attached to a bio-chip and a measurement unit is uniformly maintained, measurement precision with respect to the bio-material can be enhanced.

Also, in the bio-chip module according to the embodiments of the invention, since the bio-chip is made of a material other than a glass material, unit manufacturing costs thereof can be reduced and the handling thereof can be facilitated.

In addition, in the device for measuring a bio-chip according to the embodiments of the invention, since deformation of the bio-chip that may occur in measuring a bio-material is minimized, the selection of available bio-chips can be widened.

While the present invention has been shown and described in connection with the embodiments, it will be apparent to those skilled in the art that modifications and variations can be made without departing from the spirit and scope of the invention as defined by the appended claims.

1. A bio-chip module comprising:
a first substrate including a plurality of projections formed on a first face thereof and made of a plastic material,
the plurality of projections having a bio-material attached thereto; and
a second substrate including a plurality of through holes into which the projections are inserted, the second substrate being combined with the first face of the first substrate to form a tight attachment between the first substrate and the second substrate.

2. The bio-chip module of claim 1, wherein the second substrate is made of a metallic material or a ceramic material.

3. The bio-chip module of claim 1, further comprising a third substrate installed on a second face of the first substrate such that the first face of the first substrate is tightly attached to one face of the second substrate.

4. The bio-chip module of claim 3, wherein the second substrate is made of a metallic material or a magnetic material, and
the third substrate is made of a material different from that of the second substrate, among the metallic material and the magnetic material.

5. The bio-chip module of claim 3, wherein the third substrate includes a recess formed on one face thereof and allowing the first substrate to be inserted thereinto.

6. The bio-chip module of claim 1, wherein each of the through holes has a sloped portion on an edge thereof.

7. A device for measuring a bio-chip, the device comprising:
a bio-chip module including a first substrate including a plurality of projections formed on a first face thereof, the plurality of projections having a bio-material attached thereto, and a second substrate installed on the first face of the first substrate and including a plurality of through holes into which the projections of the first substrate are inserted; and
a measurement unit measuring the bio-material attached to the projections.

8. The device of claim 7, wherein the bio-chip module further comprises a third substrate installed on a second face of the first substrate such that the first face of the first substrate is tightly attached to one face of the second substrate.

9. The device of claim 8, wherein the second substrate is made of a metallic material or a magnetic material, and
the third substrate is made of a material different from that of the second substrate, among the metallic material and the magnetic material.

10. The device of claim 8, further comprising a flattening member evenly distributing pressure of the third substrate on the first substrate.

11. The device of claim 7, further comprising a pressing unit pressing the second face of the first substrate such that the first substrate is flattened in a state of being combined with the second substrate.

12. The device of claim 7, further comprising a prop member supporting the first substrate such that a pre-determined distance is maintained between the bio-material attached to the projections and the measurement unit.
